(19) United States
(12) Patent Application Publication
(10) Pub. No.: US 2021/0373342 A1
(43) Pub. Date: Dec. 2, 2021

(54) AUGMENTED REALITY GLASSES FOR INDICATING GEAR POSITION AND/OR OTHER INFORMATION TO A BICYCLE RIDER

(71) Applicant: Brian Gordaychik, Columbus, NJ (US)

(72) Inventor: Brian Gordaychik, Columbus, NJ (US)

(21) Appl. No.: 17/333,767

(22) Filed: May 28, 2021

Related U.S. Application Data
(60) Provisional application No. 63/033,353, filed on Jun. 2, 2020.
(60) Provisional application No. 63/060,048, filed on Aug. 1, 2020.

(51) Int. Cl. G02B 27/01; H04B 1/3827; G06T 11/00

(52) U.S. Cl. CPC G02B 27/0172; H04W 84/10; G06T 11/00; H04B 1/385

(57) ABSTRACT

Eyewear for use by a rider of a bicycle may comprise a wireless personal area network (WPAN) transceiver configured to associate with at least one electronic shifting component of a bicycle and determine a number of front and rear gears of the bicycle. The WPAN transceiver may be configured to receive an indication of a gear change of the bicycle. A processor may be configured to indicate to a user of the eyewear, based on the indication of the gear change of the bicycle, a change in gear position.

### CROSS-REFERENCE TO RELATED APPLICATIONS

This application claims the benefit of U.S. Provisional Application No. 63/060,048, filed Aug. 1, 2020 and U.S. Provisional Application No. 63/033,353, filed Jun. 2, 2020, the contents of each of which are incorporated herein by reference.

### BACKGROUND

Mountain biking involves significant climbing and descending and thus requires frequent gear changes. A mountain bike rider may be caught off guard when an unanticipated steep incline seemingly arises out of nowhere. Further, significant uphill climbing often follows downhill periods of which a rider may be in a gear that is completely unsuitable for the uphill climb which is about to follow. Often, the rider may not recall what gear was last used by the time the rider advances to the base of an uphill climb. If the rider is in a hard gear, the bike may stall as significant shifting may be required to reduce the pedaling difficulty and increase cadence. If the rider is in too easy of a gear, spinning may occur thus wasting the rider's energy. Methods and systems disclosed herein indicate to a rider the current gear position without the rider having to take his or her eyes off of the trail.

Previous attempts to provide cycling glasses have typically included features that have already been accomplished on traditional bicycle computers, for example, including cadence, speed, time of day and the like. These metrics are great to have, but leave more augmented reality and controls to be integrated into AR glasses for cycling.

### SUMMARY

Eyewear for use by a rider of a bicycle may comprise a wireless personal area network (WPAN) transceiver configured to associate with at least one electronic shifting component of a bicycle and determine a number of front and rear gears of the bicycle. The WPAN transceiver may be configured to receive an indication of a gear change of the bicycle. A processor may be configured to indicate to a user of the eyewear, based on the indication of the gear change of the bicycle, a change in gear position.

### BRIEF DESCRIPTION OF THE DRAWINGS

A more detailed understanding may be had from the following description, given by way of example in conjunction with the accompanying drawings, wherein like reference numerals in the figures indicate like elements, and wherein:

FIG. 1A illustrates a pair of glasses having an upper frame portion indicating a clear/translucent gear indicating mode or base polymer color gear indicating mode;

FIG. 1B illustrates a pair of glasses an upper frame portion having a 50% gray mode;

FIG. 1C illustrates a pair of glasses having an upper frame portion having a black mode;

FIG. 2 illustrates a pair of glasses in which tick marks indicate positions indicative of where the chain lies;

FIG. 3 shows riders field of view in which a group of riders is visible within;

FIG. 4 is a circuit diagram of circuitry for integrating with or inside glasses worn by a user;

FIG. 5 illustrates a field of view superimposed with heads up display as seen by a user;

FIG. 6 illustrates an embodiment in which on screen numbers appear above a rider's hands or alternatively in a different place/position;

FIG. 7 shows an embodiment in which gearing is displayed in a circular fashion; and

FIG. 8 shows illustrations 800 indicating that desired shifts may be indicated by finger movements/positions picked up via camera mounted on the glasses.

### DESCRIPTION OF PREFERRED EMBODIMENTS

While descending down a hill at a high speed, a rider may need to pay careful attention to rocks and/or roots and may not want to take his or her eyes off the terrain in order to look down at a computerized device. However, the rider may need to know information about the state of his bicycle and/or electronic components. For example, a rider may need to determine gear position or ratio.

Methods and systems for determining gear position are disclosed herein. Additionally, other parameters of a bike and/or components may be determined and displayed to a user using the embodiments disclosed herein.

In an embodiment, a color scheme may be used to indicate gear position. A typical mountain bike has 1 or 2 chain rings in the front and between 10 to 12 chain rings in the rear (cogs). It may be relatively obvious to know whether one is in the first or second ring of the front chain rings, however knowing which cog the chain rests on in the rear is more of a problem.

In an embodiment, glasses may be used to indicate the cog in which the chain rests on. For example, a rear cassette with 11 cogs may have 9 cogs associated with one or more colors, while the two cogs with the most teeth, i.e. the easiest cogs, are not associated with a color. The easy cogs may not be associated with a color for the reason that hill climbing with the easiest cogs may not be an issue, at least not for the beginning of a hill. Or alternatively, the display indication may be limited in colors or contrast. For a 11-51 cassette, example color and number of teeth on a cog are shown in Table 1. Other colors or color combinations may be selected for each cog.

TABLE 1

| | Number of |
|---|---|
| Color | teeth on cog |
| | 51 |
| | 45 |
| Red | 39 |
| Purple | 33 |
| Blue | 28 |
| Green | 24 |
| Yellow | 21 |
| Orange | 18 |
| White | 15 |
| Grey | 13 |
| black | 11 |

A grayscale system could also be used, wherein the color gets more black or less black per gear. For example, Table 2 may be used to indicate the cog. Another color may also be used.

TABLE 2

| | Number of |
|---|---|
| Color | teeth on cog |
| Transparent/no color | 51 |
| 10% gray | 45 |
| 20% gray | 39 |
| 30% gray | 33 |
| 40% gray | 28 |
| 50% gray | 24 |
| 60% gray | 21 |
| 70% gray | 18 |
| 80% gray | 15 |
| 90% gray | 13 |
| black | 11 |

Any color may be configured in the grayscale embodiment. For example, the color red may begin pink and turn red or purple as the number of teeth increase. Other colors, for example, green or blue may be employed in a similar manner.

A user may programmatically indicate which color to employ per cog via computer, cell phone, tablet, personal digital assistant, music player or the like. For example, a user may choose to use three colors, for example, red, green or blue, for the 3 most difficult gears. The remaining gears may be transparent or base color of a polymer or film used to indicate the color.

TABLE 3

| | Number of |
|---|---|
| Color | teeth on cog |
| transparent | 51 |
| transparent | 45 |
| transparent | 39 |
| transparent | 33 |
| transparent | 28 |
| transparent | 24 |
| transparent | 21 |
| transparent | 18 |
| red | 15 |
| green | 13 |
| blue | 11 |

Most typical bike parameter measurements, cadence, miles per hour, wattage, etc. are good to know on a long bike ride. However, the current gear(s) which the chain is resting on is more important to know instantaneously and more often. These other parameters may also be indicated, for example, using color changing polymers or via heads up display of the glasses in a periodic manner or as is necessary.

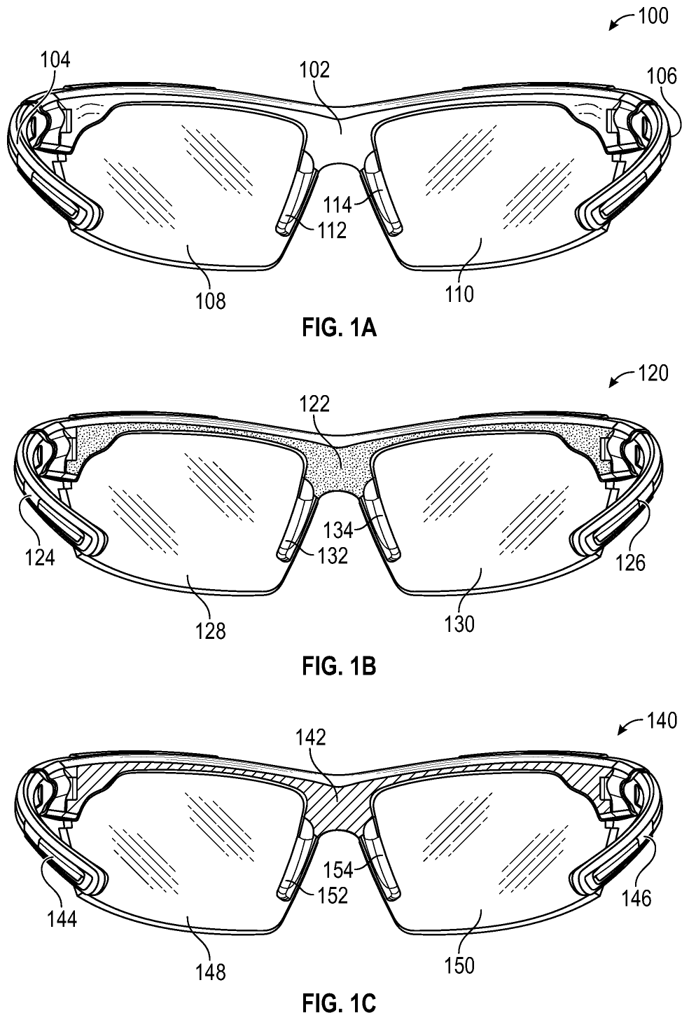

FIG. 1A illustrates a pair of glasses 100 having an upper frame portion 102 indicating a clear/translucent gear indicating mode or base polymer color gear indicating mode. Gearing may be indicated using various different colors. Glasses 100 include temples 104, 106, lenses 108, 110, nose pads 112, 114. Nose pads may include circuitry configured to indicate output conditions, for example, by vibrating.

FIG. 1B illustrates a pair of glasses 120 an upper frame portion 122 having a 50% gray mode. Gearing may be indicated using various different colors or grayscales. Glasses 120 include temples 124, 126, lenses 128, 130, nose pads 132, 134.

FIG. 1C illustrates a pair of glasses 140 having an upper frame portion having a black mode. Black color may indicate a last gear, for example, a most difficult gear or a least difficult gear. Glasses 140 includes temples 144, 146, lenses 148, 150, nose pads 152, 154.

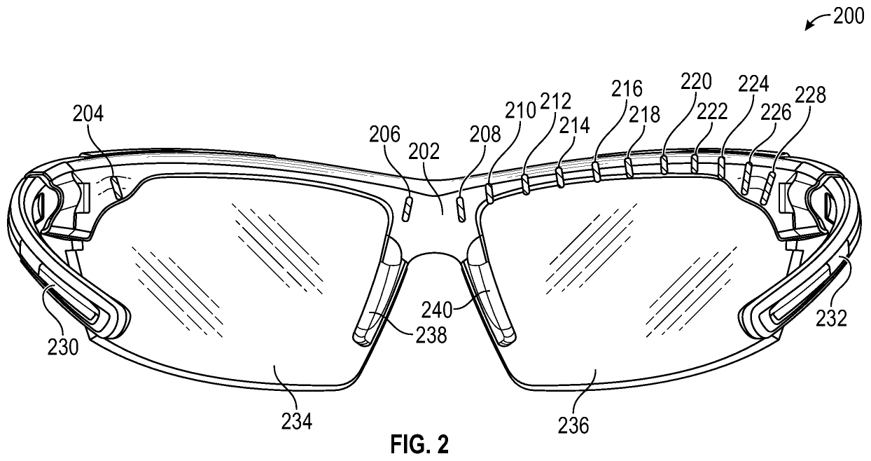

FIG. 2 illustrates a pair of glasses 200 in which tick marks indicate positions indicative of where the chain lies. In the example shown in FIG. 2, two positions 204, 206 may be indicated on the left hand side to indicate front gear position, while eleven positions 208 are shown the right hand side to indicate rear gear position. Each one of the dark tick marks may be illumined or colorized when the chain is in a corresponding position. Tick marks may be indicated in the frame portion or indicated in the lens portion using a heads up display or other means. Glasses 200 includes a frame 202, temples 230, 232, lenses 234, 236 and nose pads 134, 136. Tick marks may be illuminated by light emitting diode (LED) or other means. Tick marks may be located within lenses or on frame.

Other types of glasses may be used as well, for example, full frame glasses or glasses with smaller frames. In this way, a user may select a frame type which provides less or more of an indication of gear position. A design of the glasses/frames of FIG. 1A-1C are shown in D673,205 to Tifosi Optics Inc. In an embodiment, frame may have removable portions which change color.

Using a heads up display, a display of a gear position may appear when a touch is made on a gear shifter. In an embodiment, a touch may be a two finger touch, a thumb touch, for example, a thumb hold. The touch type selected may differ from an ordinary touch type used to signal gear change via electronic shifters. For example, a touch may be different than a button press. The touch type used to display the gear indicator may be on another touch sensitive area as the shift touch sensor. The gear display indicator may be timer operated, for example, after the touch display is detected, it may be displayed for a configurable time period, for example, 1 or 2 seconds. A touch applied to a left shifter may toggle the shifter heads up display menu, while a touch applied to the right shifter may toggle another menu. The same may be true for an up shift vs. a down shift. Touching each shift type may toggle one of various heads up display types.

Shifts may be indicated via a user indicated command which indicates a single gear changes or a different command which indicates multiple gear changes. For example, by tapping quickly, a user may indicate a multi gear change in which a derailleur may move X gears before stopping. The derailleur may be in communication with a sensor/transceiver on the pedals or crankset to ensure that the rider is pedaling during movement to ensure that the chain moves with the derailleur. In another embodiment, a finger may be held in a certain position to indicate the multiple gear change.

Glasses which do not employ frames surrounding the optical elements may also be used. Instead, color changes and/or tick marks may be noted on the optical elements themselves. The same may be true with framed glasses.

Glasses and/or frame portion may also comprise circuitry for coupling a wireless personal area network (WPAN) or wireless local area network (WLAN) transceiver for interfacing with a bicycle or bicycle components. The circuitry may instruct the change in color upon receiving a transmission from the bicycle that a shift in gear has been made. The transmission may indicate a particular gear by number or code, or the transmission may simply indicate an up/down change from the existing position. Frame portion may have a port for receiving a charging cable and/or connecting cable, for example, a USB cable. The USB or other cable may charge a batter stored in the glasses.

In an embodiment, a color change may be applied using a rapid color change polymer, for example, a polydiacetylene polymer. A heating element may be employed within the frame of the glasses to enable the change. In other embodiments, magnetic iron oxide nanostructures may be employed to rapidly change color. Other embodiments employ a Liquid-crystal display (LCD) or light emitting diode (LED) structure within the frame or within the lenses.

In an embodiment, each side of the glasses may comprise different color changing material and/or circuitry which changes the side separately from the other side. In this way, a user may be able to quickly determine the front gear position and the rear gear position using two different colors simultaneously.

Electrochromic polymers are already being used in sunglasses, however, their use is with respect to changing the color of the optical portion to, for example, block sunlight or UV rays. For example, U.S. Pat. No. 7,874,666 discloses electrochromic (EC) materials which can change their color when an electrical potential is applied, due to electrochemical oxidation and reduction reactions occurring within the materials. EC polymers are promising materials for use in sunglasses. EC polymer based devices (ECDs) exhibit several desirable characteristics. They require power only during switching state; their operating voltages and energy consumption are low; they have rapid response times.

A user may initiate an association procedure between the glasses and an electronic gear switching mechanism of a bicycle to associate the glasses with the bicycle. For example, the glasses may have a button which when pressed, may cause an association request message to be transmitted and capabilities of the glasses indicated. An association response may be received with bicycle capabilities, for example, the number of gears, gear ratio and the like of the bicycles electronic gear switching components. In an embodiment, a manufacturer or component code may be indicated for the pairing. Glasses may be coupled with a cellular phone or computer for receiving a software upgrade or software instructions for mating with bicycle components. The same may be true for the bicycle components including controllers, derailleurs, shifters, cranksets, crank arms, frame integrated computers etc. Software upgrades may be performed automatically based on a key, for example, obtained via a physical purchase of another device. For example, if a user upgrades a derailleur, the derailleur may have embedded a cryptographic key used to unlock or obtain a software portion for used on an AR glasses or other piece of electronic equipment. This may relate to other bike components, regardless of whether the components are for stationary or non-stationary bikes, whether MTB or road or other bikes etc.

Association may comprise an autodetection of a number of gears on front and/or rear of a bicycle. The display may automatically adjust the spacing of gearing indicators for display on the heads up display to match a number of gears. For example, if only one gear is employed on the front, there may be no need for a gear display of the front gear. If there are two, the spacing may accommodate two indicators in combination with a number of indicators on the rear gear.

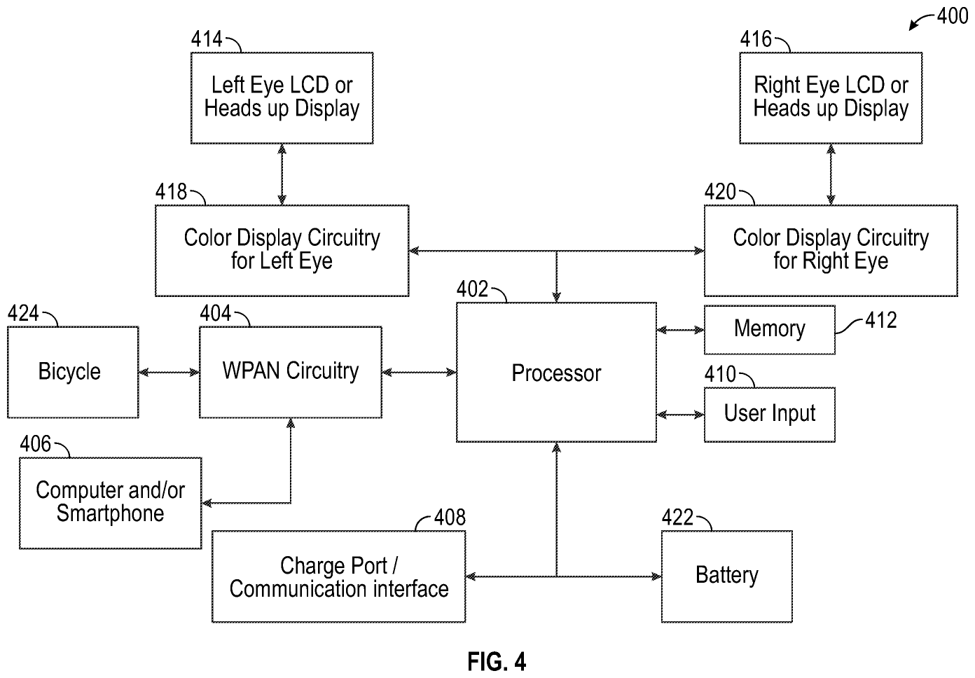

FIG. 4 is a circuit diagram 400 of circuitry for integrating with or inside glasses worn by a user. FIG. 4 illustrates a processor 402 which may be a general purpose processor configured to execute instructions resident in memory 412. Instructions may be received via WPAN circuitry 404 and computer/phone 406 or via charge port communication interface 408. User input 410 may be provided in the form of a push button interface or via phone/computer input 406 via the WPAN circuitry 404. In addition to WPAN circuitry 404, the glasses may have a cellular transceiver and/or WiFi transceiver. The glasses may comprise circuitry to detect speed, for example, cycling speed or walking jogging speed of a bicycle 424. This may allow the glasses to report and participate in vehicle 2 everything (V2X) and pedestrian 2 everything (P2X) communication. A phone may act as a relay, for example, for ANT or ANT+ commands/messages.

A user will likely be riding with AR glasses coupled to or interfacing with a phone. In some embodiment, both devices may have cellular circuitry and the two devices may negotiate as to which device will act as a primary V2X communication device. This way, both devices are not actively communicating V2X as this would be a waste of resources/battery. In an embodiment, a phone may act as a V2X relay for the AR glasses and/or bicycle.

In an embodiment, a phone or AR glasses may receive ANT commands from a bicycle and may interpret those commands for transmission over a V2X interface. For example, an ANT command may be received by the AR glasses which indicates that the rider is applying brakes. The ANT command may be processed by the glasses and a V2X data may transmission may indicate braking. The V2X data may be transmitted over the V2X link to another rider, a vehicle or a ground based transmission/reception point.

Similarly, a crankset may perform much the same function. A crankset may determine that a power increase is being applied, and may indicate an increase in speed or an initiation in movement over the V2X link. In the same way, an ANT command may be processed and a V2X data may be provided.

The V2X command may indicate, for example, the brake or power increase commands discussed above or any other command or information element disclosed herein. In an embodiment, the V2X data may be received by the AR glasses so as to indicate which rider (of a group of one or more riders) is braking or increasing power. Rider position may be indicated in one or more broadcast, multicast or unicast packets transmitted by the rider.

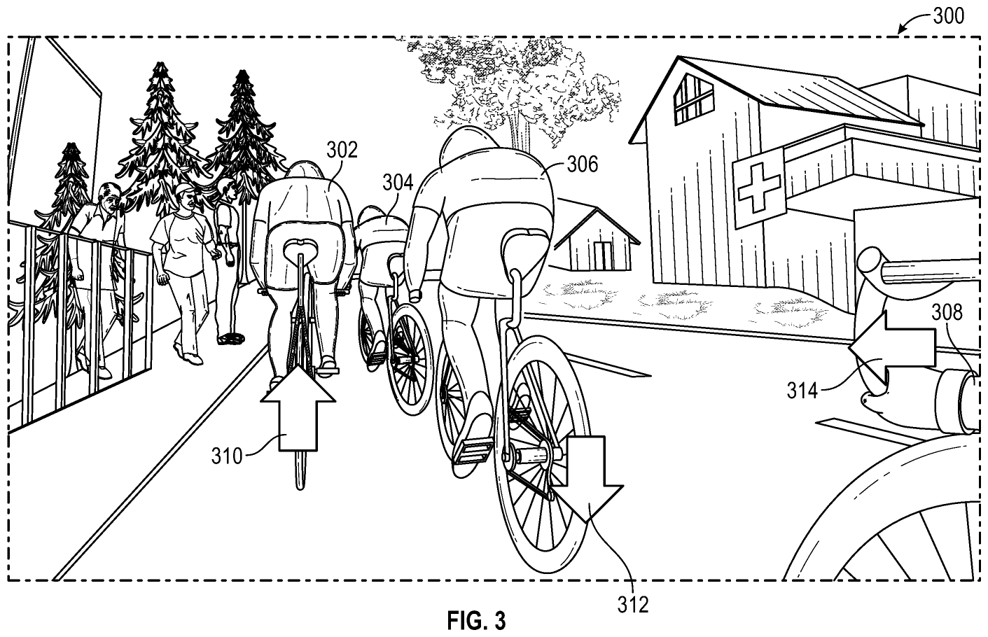

FIG. 3 shows riders field of view 300 in which a group of riders 302-308 is visible within. Arrow 302 indicates that rider 302 has begun to increase his power, while down arrow 312 indicates that rider 312 has tapped his brakes. The rider on the far right 308 is shown with an arrow 314 indicating a left potion meaning this rider 308 is turning closer. This left turn may be indicated by an ANT motion sensor in the handlebars. V2X is used herein as an example, for at least the reason that detecting these riders and their ANT commands may require pre-registration and setup between ANT devices. By genericizing the ANT commands into, for example, V2X data, a receiver may parse the ANT command (or information corresponding to it) via V2X. A rider's other information, for example, motion information, transmission power, transmission angle or the like, to determine which bicycle is the one which is braking, turning, increasing power or the like may be determined via the V2X data. Other information may be displayed on the AR, for example, shift gestures. Display of an arrow is shown by example only. Other information symbols or textual information may be used. In an embodiment, object detection methods and devices of U.S. Pat. No. 9,671,785 disclosed by reference herein may be employed.

Information received at the AR may be anonymous. For example, V2X transmission devices may chose to indicate parameters based on a privacy level or location level. In a privacy embodiment, bicycle information/parameters may only be transmitted to friends or members which are connected in a social network. At a location level, bike parameters may be indicated based on a location. For example, if another bicyclist is within range, for example, 30 feet, 60 feet, 100 feet or the like, information may be provided. Distance may be determined by signal strength, by users within a given cell, using V2X parameters or the like.

For example, an ANT frame indicating a particular bike parameter may be received from a bicycle component by a phone or AR glasses. The command may be processed according to an application specific protocol, for example, Shimano DI2 or other protocols, to determine the type of command. That command may be generalized into a (stopping, accelerating, increasing, etc.) parameters and transmitted along with other information herein in a single packet or a plurality of data packets over V2X. In this way, two devices may support a same capability of receiving the generalized packet even though both end devices do not support Shimano DI2. The generalized packet type may be associated with an application running on a smartphone or AR glasses.

In an embodiment, a communication interface may include an Adaptive Network Topology (ANT) interface, a Wi-Fi interface, cellular interface, Bluetooth interface etc. In one embodiment, ANT messages may be encapsulated and transmitted over a Wi-Fi or cellular interface for ANT processing at an ANT capable remote server or MEC server. In this way, the local devices (local AR glasses, phone, bike etc.) may not have to process ANT messages, but rather these messages can be passed directly to a MEC server which processes responses. The phone, glasses or bicycle may receive ANT messages via cellular and may provide the ANT messages to other components for processing.

ANT has a maximum data rate of only 60 kilobytes per second which is too slow for many advanced augmented reality features. Therefore, the glasses should use ANT for interaction with the bicycle electronics, but then use Wi-Fi or cellular for video/audio communication. In an embodiment, a device may receive an ANT message with an indication to wake up another radio, for example a Wi-Fi radio. The ANT messaging may include parameters for the Wi-Fi radio to use to access a network, receive information, connect to a server or the like.

When an ANT transmitter determines a need to transmit at a higher power or higher modulation and coding scheme (MCS), a switch to a different MCS may be indicated in the ANT transmission, for example, in a preamble or a following data portion.

The processor may be configured to communicate with circuitry for driving the color changing elements, for example, magnetic elements or temperate change elements. The processor 402 may also communicate with an LCD or heads up display elements which may be integrated within or on the lens portion(s) of the glasses. Each display for each eye (left) 414 and (right) 416 may have separately drivable controls 418, 420 for color change and/or LCD illumination. Color change may be indicated via LCD or heads up display in addition to or in place of polymer type color change of the frame.

Battery 422 may be charged via charge port 408 and/or may be charged via wireless methods. In one embodiment, battery may be charged while the glasses are coupled to a compatible bicycle. Thus, the same charger used to charge a battery of bicycle electronic components may be used to charge glasses. Eyewear may also comprise a camera for recording a position of the handlebars. In this way, the camera may detect handlebar position with respect to a user's field of view. The heads up display may place an indicator (for example, a numerical indicator) over each hand to indicate gear position.

A WPAN 404, WLAN or cellular transceiver may offload data processing to a cellular phone or to a cloud device. A microphone and/or camera may be used to detect problems with the bicycle. For example, a microphone may detect that a disc brake is rubbing and may suggest an appropriate repair technique to the operator. Additionally, a camera may be used to analyze the bike for other problems and suggest corrective action. The glasses may display the suggestion of corrective action overlaid over the part which may be defective or the screw or other member which needs to be adjusted/tightened etc. The WLAN or cellular transceiver may aid in this process, for example, by sending comprising the audio/video recorded by the camera/mic and receiving messages comprising the suggested repairs.

In embodiments, the color changing device may be located on the frame at an upper portion of the lens, at a lower portion of the lens or at any other portion which is within the user's peripheral field of vision. Ideally, the color change indicator will not obstruct a field of view, but will also not be outside of an average user's peripheral field of view. In this way, a user may not have to move his or her eyes to determine the color. Alternatively, in an embodiment, a user may have to adjust his or her eyes to determine the color. For example, a user may have to look up, left, right or down to view the color or indicator. Ideally, the color change may be subtle enough to not be noticeable to a user unless the user desires to determine the color.

In embodiments, a color may not necessarily be used to indicate gear position. A series of illuminated dots or lines may be used. A presence or absence of an indicator may be used alternatively or in combination.

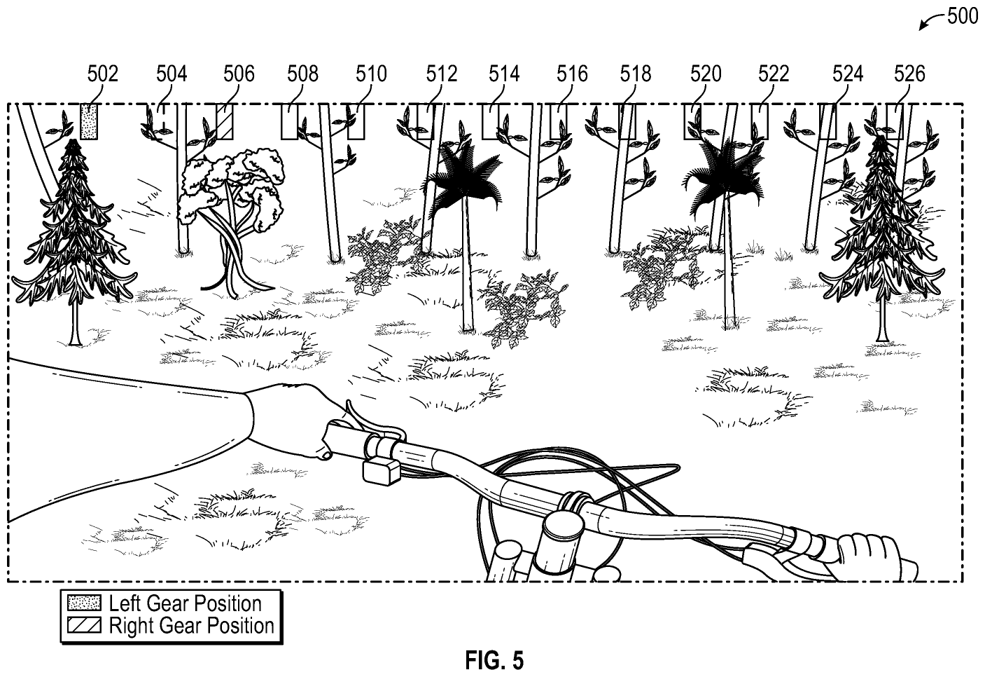

FIG. 5 illustrates a field of view 500 superimposed with heads up display as seen by a user. FIG. 5 illustrates an embodiment where a gear which is selected is shown in a solid indicator or other type of indicator, while unselected gears are shown in boxed indicators (or may not be shown at all in some instances). In this example, the left most indicator 502 shows the front gear, while indicator 506 shows selection of the rear gear, i.e. in this case a first of 11 rear gears is selected. Indicator 504 shows an unused front gear and indicators 508-526 illustrate unused rear gears. In this case, an easiest front gear is shown selected and an easiest rear gear is shown selected. The display shown in FIG. 5 may be constantly displayed or activated upon triggering by a touch button. Boxed out portions which indicate gears which are not selected may or may not be displayed depending on user preference.

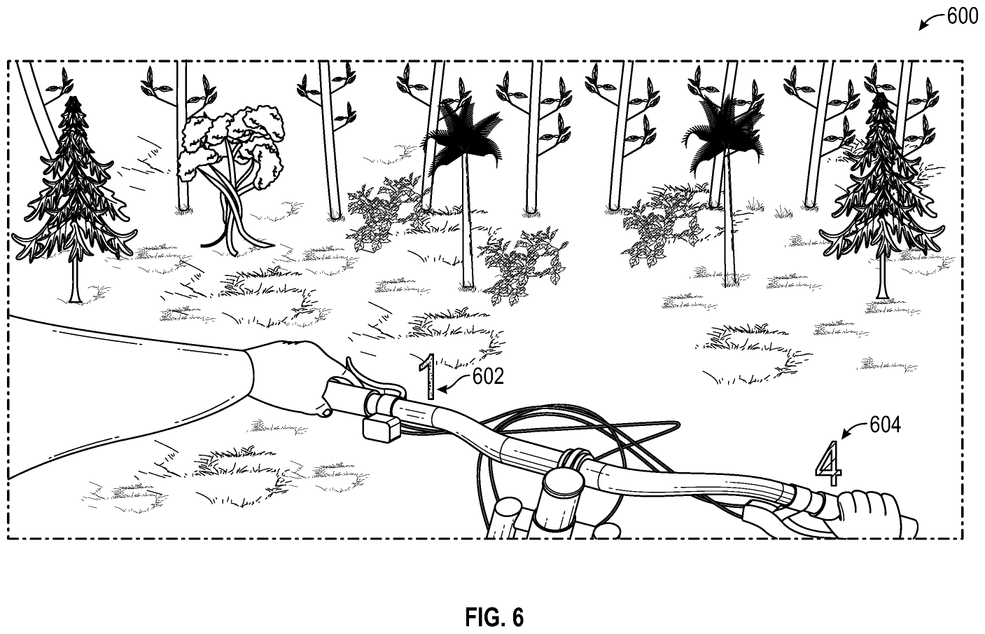

FIG. 6 illustrates an embodiment 600 in which on screen numbers appear above a riders hands or alternatively in a different place/position. In this embodiment, a camera and/or sensor(s) are mounted on the glasses such that the handlebars and hands of the rider are tracked. In this case, an easier of two gears 602 is selected up front, and a 4th of 11 gears 604 is selected in the rear. In embodiments, the numerical indications may appear on the handlebars themselves, or on another device.

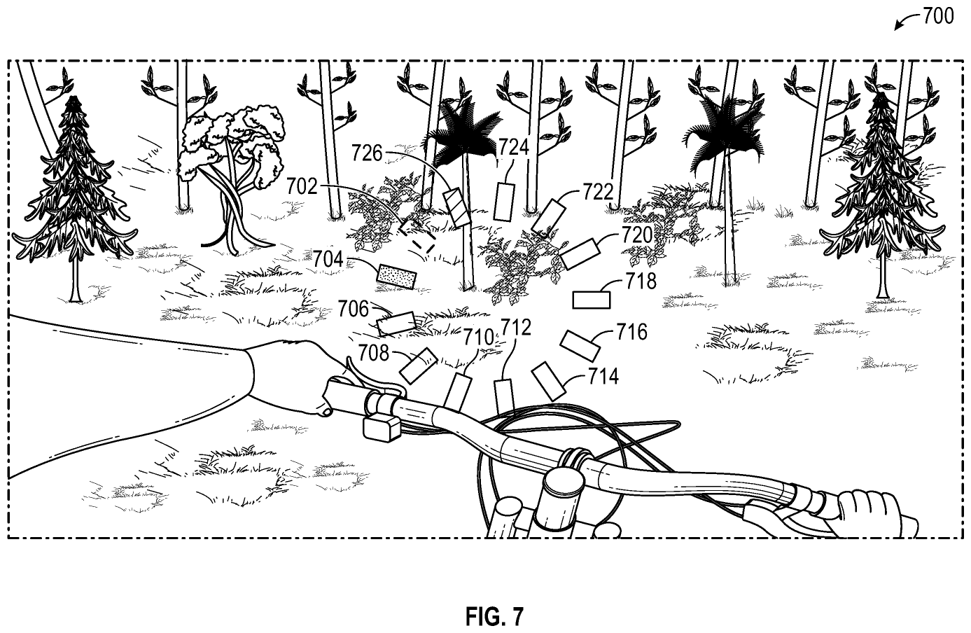

FIG. 7 shows an embodiment in which gearing is displayed in a circular fashion 700. In an embodiment, the number of gears displayed for the front and rear are selected based on bicycle capabilities in accordance with information gathered during the association procedure. In this example, there are two front gears and 11 rear gears. In the front, a second of two gears is used and indicated 704, while in the rear, the 11th gear is used and indicated 726. Unused gears are shown via display boxed 702 and 706-724 which are not filled.

FIGS. 5-7 show rectangular indications, but other indications may be used as well, for example, circular or other patterns of shapes.

In an embodiment, the heads-up display may appear following a first shift and remain for a time duration. In this way, as a rider begins an assent up a hill, the rider can easily ascertain a number of additional shifts required to get into a desired gear. The heads up display may appear just after a rider slows to a stop. This may indicate a gear position, for example, when the rider is sitting at a red light or stop sign. In embodiments, a camera and/or accelerometer may detect that the rider is stopped and may then indicate gear position so that the rider has the ability to shift higher or lower at or before the time of acceleration from a stop position. In an embodiment, the rider may be determined as in a stopped position when a dominant foot (or any foot) is no longer clipped into a pedal and/or the bicycle is motionless. If the rider moves one of the crank arms backwards (indicating getting ready to take off), the gear display may be provided to the user.

In other aspects, it is often important to a rider to understand a position taken at a previous uphill ascent or downhill descent. For example, a terrain may be rooty (i.e. many tree roots exposed above the dirt) or rock (i.e. many large and/or jagged rocks exposed) which makes an ascent or descent challenging to negotiate. A position taken on the trail may be recorded at a higher accuracy than GPS, for example, via camera and position measurements.

In an embodiment, AR glasses may make record, via a camera or other device, of the location, position and/or wheel location of the rider's bike. This way, before a next ascent, the rider can view in real time a position taken up or down the hill. The view may be provided to the rider as an augmented overlay on top of the user's view of the hill. AR glasses may determine whether a front wheel or rear wheel hit the ground first on a jump, via sensors in the wheels or tires. The same sensors may provide tire pressure information or other information. These sensors may also determine where a body weight is located, for example 45% rear vs 55% front etc.

The AR glasses may comprise a camera and/or sensors such as GPS or other location based sensors for computing/determining location. The glasses may also rely on a cell phone for some or all of the location calculation. In an embodiment, the glasses may have a low resolution camera to provide the wheel position back to the phone which then stores wheel position information in accordance with time information. The phone or other wireless mobile device may then indicate location information to the glasses for determining when to present information to the rider.

Similar embodiments may apply to road biking, running or other sports. For example, many road bike riders will ride a same course, for example, a same path from the rider's home out through the country and back to the rider's home multiple times in a week, month, year etc. It would be interesting for the rider to be able to learn, during the ride, where the rider stands time wise in terms of previous rides. In an embodiment, the AR glasses may display an overlay of where the rider was at a last ride. If the rider was ahead in time previously, the glasses may display an image of the rider out ahead. If the rider is behind, the glasses may simply display an indication of the previous ride being behind, i.e. the rider is now riding faster/fastest. The same embodiments may apply for running, especially for track distance running where it would be ideal to know how far (in distance or time) behind a best or last run the runner was.

In an embodiment, display of the rider out ahead may be an augmented reality display of an actual picture or simulated image of the rider taken from a photograph or video. In other embodiments, it may simply be a stock image or an image selected by the user. As the users gains on the previous time, for example, the image representative of the past ride may be adjusted closer until at such point the current ride is at or exceeding the previous time. Then the image may be removed from the display and an indication of “fastest time” or “rider back” could be displayed or indicated.

In an embodiment, a rider displayed from a previous ride is not continually displayed, but is rather displayed periodically. For example, as a rider comes into a field of view, for example, the rider in view is at a bottom of hill, and the rider with AR glasses has just reached the peak of this hill, the rider at the bottom of the hill will be displayed so long as a distance between the two riders is being closed. Additionally, if a rider with AR glasses looks down or away from the position of which a forward rider is displayed, that forward rider should be removed from view. This may occur with an accelerometer, sensors, camera, etc.

AR glasses may be configured to fill in portions of a display environment based on stock images or previous images/videos taken by a rider. For example, a pine barrens ghost town might be displayed as it looked in the 1700 or 1800s based on an artist's rendition overlaid over other remnants of the real environment as the rider views the environment. In other examples, if it is foggy or cloudy and a certain video is impeded, the glasses may fill in the view from previously recorded images taken by other riders.

Record performances may also be displayed. For example, 1972 Eddy Merckx set a new hour record, i.e. the record for the longest distance cycled in one hour on a bicycle from a stationary start. The glasses may display hour record information from certain users which are stored in an online database. A phone may provide requests/responses for information in the database. An image of a default user or a custom image of a user may be displayed in an AR or other fashion in accordance with that user's position.

Similar display information may be made for weight lighting by recording weights picked up and lifted by a user.

In embodiments, the glasses may automatically curtail certain information, based on a riders detected cadence, speed, altitude, location, bearing, wattage, obstacle detection or any other parameter herein. For example, if a rider is riding at a speed above a threshold and the rider is in a city environment, display information may be curtailed until a speed drops below a threshold. The speed threshold may be lower if the rider is in a trail environment which is denoted as more technical of a trail. If the rider is riding on a rail trail, for example, where the terrain may be non-technical in nature, less stringent thresholds may be configured for curtailing display of certain information. In an embodiment, a sensor mounted on the bicycle suspension may indicate terrain conditions and thus the AR glasses may curtail information based on the detected terrain. Certain information may be curtailed when a bicycle is operating in a pedal assist mode, for example, based on whether or not the user is peddling as opposed to allowing the motor to perform substantial work.

By curtailing information, the AR glasses may limit the intensity of the augmented display information. Additionally, information may be limited to a certain portion of the display screen. For example, a gear position may be limited to the upper and outer portions of a user's field of view when a threshold is met as opposed to a more central area when the threshold is not met. Thresholds may be user configurable or may be set based on default settings. Curtailing thresholds may be set based on activity information, which may be derived via camera, speed or other sensors and/or sensor information received from other devices including bicycle, shoes and/or other devices. For example, the AR glasses may detect that a user is running based on the camera detecting running motion vs. walking motion. Feedback may be provided via a treadmill, stationary bicycle or other device for example.

In an embodiment, the rider or other user may be provided with arrow notations indicating whether to take a more left or right position. A suggested gear position may also be provided. For example, if a rider successfully took a climb in a smaller or larger gear than is currently being utilized, the glasses may suggest an alternative gear.

Embodiments disclosed herein may also relate to competitive or team based activities. For example, in a race simulation, instead of displaying a user's past performance information, the race competitors and/or competitor information may be displayed on the glasses.

In an embodiment, a camera on the glasses may be used to detect movement of a user's finger or wrist to detect an indication to shift gears. The camera may be mounted on glasses and transmit information to components of a bicycle.

Alternatively, or in combination, cameras and/or sensors may be mounted on the bicycle. For example, a movement of the thumb or other digit on the left hand may signal a shift to the front chainring, while a movement of a thumb or other digit of the right hand may signal a shift of the rear chainring. A thumb movement may signal a downshift, while a pointer finger or index finger movement signals an upshift (or vice versa). Finger movements may be programmed, by a user, via a computer or smartphone which is remote from the glasses. The glasses may be communicatively coupled to the bike electronic shifting components and/or the smart phone (which may be coupled to the bike electronic shifting components.

By removing conventional shift levers, as well as required cabling and wiring, an added weight of the camera integrated into the AR or other electronic glasses may justify the higher cost or added weight of the glasses. In an embodiment, a single finger extended on one hand may initiate the shift, while the circuitry may not shift if an entire hand (or both) is/are moved off the handlebars. There may or may not be no shift if the users hands are taken off the bars even though a finger position or movement may indicate the shift.

Removal of brake levers may be too dramatic as this may be dangerous if electronic mechanisms fail. However, different finger positions may indicate a desire to brake even though traditional brake levers remain on the bike. This may also allow for electronic braking in case an object is detected, for example, a car door opens into a bike lane ahead of the rider. Instead, the AR glasses may simply flash a warning sign that the car door has opened. The AR glasses may determine this action from one or more of: an unlicensed transmission of a V2X peer to peer transmission from the door opening vehicle; a camera mounted on the bike, a camera mounted on the glasses, or any other method. This assumes that the door opening vehicle has a peer-to-peer transmitter for indicating the door opening indication.

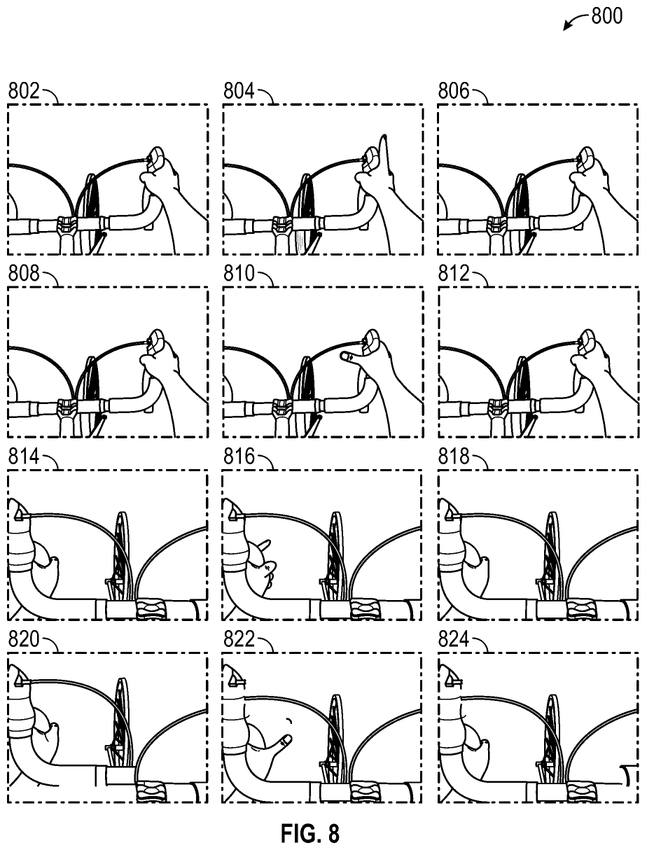

FIG. 8 shows illustrations 800 indicating that desired shifts may be indicated by finger movements/positions picked up via camera mounted on the glasses. Alternatively, or in combination, the handlebars may have sensors to sense the changed finger positions. If an entire hand is removed from the bar, no shift may be performed. However, it may be that a gesture must be completed before a shift engaged, for example, finger on bar, finger off bar to an extent/distance, finger back on bar in position. Gesture detection may be performed, for example, using methods and devices as suggested in U.S. Pat. No. 8,819,812 disclosed by reference herein.

In an embodiment, a right hand may be resting on a brake level and a thumb or pointer finger may be used to indicate an upshift or downshift of the rear gear. Any number of selected fingers, positions or movements desired by the user may be selected/programmed accordingly. For example, a movement of the thumb from on the bar to off the bar and back on the bar may cause a shift into an easier gear or vice versa. A movement of the pointer finger from on the bar to off the bar to back on the bar may cause a shift into a more difficult rear (or front gear) or vice versa. Similarly, a finger movement while the hand rests on the left drop bar may cause a gear change in the front. Similarly, the hands may be resting on the tops, i.e. the top portion of the handle bar and a movement of any finger on each hand may cause a gear shift. In this way, the user may be presented with additional shifting positions which would normally be out of reach given the users fingers were not within proximity to the gear shifter. Mountain bikes may function similar to road bike controls in this manner; however, it may be less desirable to have drop bars on a mountain bike. Stationary bikes may be supported as well.

At step 802, a user may have a right hand resting on the top handlebar, move a right pointer finger 802 in a forward position and then move the right pointer finger back to the original position 806. This may indicate a shift (e.g. downshift or upshift) of the front or rear chainring. Similarly, at step 808, a user may have a right hand resting on the top handlebar, move a thumb finger 810 in a left position and then move the thumb finger back to the original position 812. This may indicate a shift (e.g. downshift or upshift) of the front or rear chainring.

On the left side of the handlebars, at step 814, a user may have a left hand resting on the drop handlebar, move a left pointer finger 816 in a forward position and then move the left pointer finger back to the original position 818. This may indicate a shift (e.g. downshift or upshift) of the front or rear chainring. Similarly, at step 820, a user may have a left hand resting on the drop handlebar, move a thumb finger 810 in a right position and then move the thumb finger back to the original position 824. This may indicate a shift (e.g. downshift or upshift) of the front or rear chainring. For the drop bars, a sensor or camera may be located on an upper portion of the handlebar to perform the digit sensing.

One method of programming the glasses to detect desired finger movement to shift position is to input the shift type/position via a smart phone while simultaneously recording the finger movement via a camera mounted on the glasses. This may help the camera and glasses software to detect movement on various types of handlebars, for example, flat bars, drop bars, BMX style bars, etc.

In some embodiments, it may be difficult for a camera to differentiate between a shift indication (or other command indicated visually) and one which may be a subtle unintentional movement which is not indicative of a command. For example, a hand movement may be assigned to allow for a seat post to be lowered. If the hand movement is detected and a given force is applied to the saddle, the seat post may be allowed to lower. If, for example, the hand movement is detected, but the rider is not sitting on the saddle, the seat post may not lower.

In another example, a rider may need to speak a command, for example “lower,” while putting pressure on the saddle and indicating a hand command for the seat post to lower. Similarly, a voice command may be performed just prior to, during or after a hand movement to indicate a gear shift or other change to the bicycle. In an example, a user may utter the word “shift” which indicates that a shift is to follow and a hand movement may indicate a shift direction (up or down) and/or a number of gears to shift in that direction. There may be an electronic button on the handlebars, brake or shift levers that may be actuated, and while pressed, a voice command is spoken. For example, while pressing the button, the rider may voice input “down 3” or “up 4” to indicate 3 gears down or 4 gears up. A motor may then move the chain accordingly.

In embodiments, glasses may detect capabilities of the glasses, bike, and/or other components may report these capabilities to a central repository. In this way, any feature disclosed herein may be conducted or enabled based on a capability of indicated capabilities of a glasses worn by a user and glasses worn by a user in communication. For example, assume two users would like to meet and participate in, for example, voice communication during a ride. Each user may exchange capability information for participating in said voice communication exchange. If two users do not have at least some shared capability, the communication may not occur. A request and response mechanism may be employed to request and respond to capability exchanges over the internet or other network.

Glasses may wirelessly interface with the front and/or rear shock of a bicycle to detect movement and communicate to one or both of the shocks to make an adjustment. The glasses may perform this function autonomously or via command by the user. Adjustment commands may be provided wirelessly.

Some prior art applications, for example, TrailForks display trail difficulty by color, for example, black is more difficult than blue and blue is more difficult than green. However, these color coded trails do not provide a fine layer of information for one to accurately determine whether a trail is too easy or too difficult. In an embodiment, the glasses will provide more fine detail, by changing color, as more difficult or more easy trail segments are coming up.

The ability to display more detailed information on trail difficulty may be done via camera. For example, the camera may record an uphill period for a delta time period and also detect that a portion of the uphill is rocky or rooty. If so, the camera may rank that trail portion a blue+ or black−, indicating that a blue trail segment is more difficult than average or that a blue trail turns to black for a period.

The camera may also detect that logs or other obstructions are obstructing a trail. This may cause a report to be sent to a server for indicating to other users that a trail is obstructed. The accelerometer may determine that a rider has gotten off the bike, carried over the obstruction, and got back and ridden along the trail. If a number of users over a period have performed this action, then the server may reflect the obstruction to users.

Users may indicate trail conditions, for example, that a trail or trail section is wet, for example, too wet to ride. This information may be provided to others along with a date/time the information was recorded. Information about a trail segment may be displayed when a rider is near that trail segment or when the rider is expected to ride that segment, based on previous rides the rider performed and/or based on previous riders who started a ride at a location and subsequently rode that trail portion. When wet, for example, AR glasses might display a visual indication of a wet trail or an overlay of how wet a trail may be as opposed to a textual indication. Alternatively, a display might display “do not ride” when a trail is wet.

Power use of components and AR glasses or other devices of a rider may be in low power mode when not in use or when a bike is stationary. Upon detection of movement, devices may change state. Alternatively, or in combination, a button(s), voice command, or other mechanism may activate components or interfaces with a bicycle. Voice may be used to indicate any information from a rider herein. A speaker may indicate information back to the rider.

Power control for electronic shifting bicycles may be important when there is no means to perform a shift when a battery is emptied. Thus, a bicycle and/or components such as AR glasses may monitor battery power of bicycle components and indicate such a power level to the user visually. For example, the glasses may indicate power of a battery used for gear shifting as a percentage, as a number of shifts remaining, etc. In an embodiment, if the battery power level is below a threshold, the glasses or other components may disable functionality to save critical power for any shifting that may be required. For example, a lamp light may be lowered or configured to blink instead of always on mode.

In embodiments, rear and/or side facing cameras may be located on a frame of a pair of glasses. In this way, a rider display may view rear portions if necessary or if signaled to the glasses (for example, via eye movement, audio input control or via other methods).

Bicycle components and/or accessories may be powered or charged wirelessly via a power charger located on the bicycle, for example, on or inside of the bicycle frame. A battery may provide power via one or more electrical leads that provide power to a wireless power transmitter (having a transmitting coil) for transmitting power to a wireless power receiver (having a receiving coil). For example, a battery may provide power to a transmitter located inside the handlebars. This transmitter may provide power wirelessly, from inside carbon fiber handlebars, to a lamp located on top of and outside of the handlebars. Various parameters may be calculated by the power transmitter and/or power receiver and displayed on the augmented reality glasses.

For example, calibration parameters may be displayed. These parameters may indicate how well the coupling between the transmitter and receiver are. The glasses may provide information as to an adjustment to make to improve the coupling between transmitter and receiver. Additionally, or alternatively, a marker may be placed on the handlebar or other location indicating a location of the transmitting coil. If this marker is obstructed, for example, from handlebar tape, a rider may detect ideal mounting location of a receiver via an interface integrated with the glasses.

An electrical lead which supplies power and/or data transmission among components and accessories of a bicycle may be interleaved within the carbon fiber frame so as to save weight and avoid unnecessary wires. Similarly, wireless power and data transmitter/receiver circuitry may be integrated on or into the carbon fiber so as to provide removable power spots for accessories. At junctions, for example, between a frame and a fork, a relay may be used to transfer power wirelessly to a component located on the fork. The same may be true for other components and accessories of a bicycle, for example, handlebars, shifters, stem, headset, saddle, hub, spokes, tires, etc.

One of skill in the art will recognize the embodiments disclosed herein may apply to other areas, for example, fitness, sports, or other areas, which require or justify the use of electronic color changing glasses. These glasses may be used in other areas in which a color change may indicate a parameter to a user. One of skill in the art will recognize that the eyewear may comprise elements of traditional eyewear, for example, sunglass eyewear. For example, AR glasses may be mounted on a bicycle helmet or implemented as a heads up display. AR glasses may or may not have lenses.

What is claimed is:

1. Eyewear for use by a rider of a bicycle, the eyewear comprising:
a wireless personal area network (WPAN) transceiver configured to associate with at least one electronic shifting component of a bicycle and determine a number of front and rear gears of the bicycle;
the WPAN transceiver configured to receive, an indication of a gear change of the bicycle; and
a processor configured to indicate to a user of the eyewear, based on the indication of the gear change of the bicycle, a change in gear position.
